(19) United States
(12) Patent Application Publication
(10) Pub. No.: US 2002/0061297 A1
(43) Pub. Date: May 23, 2002

(54) Novel recombinant DNA vectors for gene therapy

(75) Inventors: Walter H. Gunzburg, Modling (AT); Brian Salmons, Modling (AT)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Bavarian Nordic Research Institute A/S

(21) Appl. No.: 09/965,135

(22) Filed: Sep. 27, 2001

Related U.S. Application Data
(63) Continuation-in-part of application No. 08/925,214, filed on Sep. 8, 1997.
Application No. 08/925,214 is a continuation of application No. PCT/EP96/01002, filed on Mar. 8, 1996.

(30) Foreign Application Priority Data
Mar. 9, 1995 (DK) DK 0244/95

(51) Int. Cl. A61K 48/00; C12N 7/00; C12N 7/01; C12N 15/86; C12N 15/861; C12N 15/867

(52) U.S. Cl. 424/93.21; 435/456; 435/235.1; 435/320.1

(57) ABSTRACT

The invention refers to a novel recombinant vectors useful for gene therapy of viral infections and of diseases associated with B and T cells. The present invention relates, furthermore, to novel usages of the two products of the open reading frame of mouse mammary tumour virus.

### RELATED APPLICATION(S)

This application is a continuation-in-part of Application No. 08/925,214, filed Sep. 8, 1997, which is a continuation of International Application No. PCT/EP96/01002, which designated the United States and was filed on Mar. 8, 1996, published in English, and which claims priority to Danish Application No. DK 0244/95, filed on Mar. 9, 1995.

The entire teachings of the above application(s) are incorporated herein by reference.

### BACKGROUND OF THE INVENTION

Mouse mammary tumour virus (MMTV) is a retrovirus that is associated with mammary tumorigenesis in susceptible mice (Salmons, B. and Güinzburg, W. H., Virus Res., 8:81-102, 1987). The virus is transmitted from the mother mouse to the suckling offspring via the milk. In addition to the usual retroviral genes gag, pol and env, the Long Terminal Repeat (LTR) of Mouse Mammary Tumour Virus (MMTV) contains an open reading frame (ORF) (Donehower, L. A. et al., J. Virol., 37:226-238, (1981); Kennedy, N. et al., Nature, 295:622-624 (1982)) which is highly conserved between different MMTV isolates (Brandt-Carlson, C. et al., Virology, 193:171-185 (1993)). Although ORF specific transcripts have yet to be cloned, in part due to their low abundance, a splice acceptor site has been mapped immediately upstream of the 3′ LTR which is presumed to generate putative 1.7 kb ORF transcripts (Wheeler, D. A., et al., J. Virol., 46:42-49 (1983); van Ooyen, A. J. et al., J. Virol., 46:362-370 (1983)). Recently, a novel promoter has been identified in the MMTV 5′LTR and transcripts initiating from this promoter also splice to the ORF acceptor site (Günzburg, W. H. et al., Nature, 364:154-158 (1993)), increasing the potential for diversity of ORF related products.

Two biological activities, defined by functional assays, have been ascribed to products of the ORF. One of these activities is a transcriptional repressor, Naf, which downregulates in trans expression from MMTV based constructs (Salmons, B., et al., J. Virol., 64:6355-6359, (1990); Günzburg, W. H. and Salmons, B., Biochem. J., 283:625-632 (1992)). The second activity displayed by the MMTV ORF is a superantigen (Sag) activity (Choi, Y., et al., Nature, 350:203-207 (1991); Acha-Orbea, H., et al., Nature, 350:207-211 (1991)). Expression of Sag in vivo results in the stimulation and growth, followed by deletion, of reactive T cells (reviewed in Acha-Orbea, H. and MacDonald, H. R., Trends in Microbiology, 1:32-34 (1993)). This effect is specific in that the Sag of a given MMTV variant interacts with specific classes of the twenty described V13 chains of the T cell receptor (Pullen, A. M., et al., J. Exp. Med., 175:41-47 (1992), Huber, B. T., Trends in Genetics, 8:399-402 (1992)).

The viral Sag has been shown to be a type II membrane anchored glycoprotein of 45 KDa by in vitro translation studies (Korman, A. J., et al., The EMBO J., 11:1901-1905 (1992), Knight, A. M., et al., Eur. J. Immunol., 175:879-882 (1992)). Further, Sag proteins of 45/47 kDa have also been synthesized in baculovirus (Brandt-Carlson, C. and Butel, J. S., J. Virol., 65:6051-6060 (1991); Mohan, N. et al., J. Exp. Med., 177:351-358 (1993)) and vaccinia virus (Krummenacher, C. and Diggelmann, H., Mol. Immunol., 30:1151-1157 (1993)) expression systems. This 45/47 kDa glycoprotein may require processing to a 18 kDa cleavage product (Winslow, G. M. et al., Cell, 71:719-730 (1992)). A Sag specific monoclonal antibody detects Sag expression on LPS-activated, but not nonstimulated, B cells even though the latter cells express a functional Sag. Thus undetectable levels of Sag are sufficient for superantigen activity ((Winslow, G. M. et al., Cell, 71:719-730 (1992); Winslow, G. M. et al., Immunity, 1:23-33 (1994)).

The use of retroviral vectors (RV) for gene therapy has received much attention and currently is the method of choice for the transferral of therapeutic genes in a variety of approved protocols both in the USA and in Europe (Kotani, H., et al., Human Gene Therapy, 5:19-28 (1994)). However, most of these protocols require that the infection of target cells with the RV carrying the therapeutic gene occurs in vitro, and successfully infected cells are then returned to the affected individual (Rosenberg, S. A., et. al., Human Gene Therapy, 3:75-90 (1992), Anderson, W. F., Science, 256:808-813 (1992)). Such ex vivo gene therapy protocols are ideal for correction of medical conditions in which the target cell population can be easily isolated (e.g., lymphocytes). Additionally the ex vivo infection of target cells allows the administration of large quantities of concentrated virus which can be rigorously safety tested before use.

Unfortunately, only a fraction of the possible applications for gene therapy involve target cells that can be easily isolated, cultured and then reintroduced. Additionally, the complex technology and associated high costs of ex vivo gene therapy effectively preclude its disseminated use world-wide. Future facile and cost-effective gene therapy will require an in vivo approach in which the viral vector, or cells producing the viral vector, are directly administered to the patient in the form of an injection or simple implantation of RV producing cells.

This kind of in vivo approach, of course, introduces a variety of new problems. First of all, and above all, safety consideration have to be addressed. Virus will be produced, possibly from an implantation of virus producing cells, and there will be no opportunity to precheck the produced virus. It is important to be aware of the finite risk involved in the use of such systems, as well as trying to produce new systems that minimize this risk. The essentially random integration of the proviral form of the retroviral genome into the genome of the infected cell led to the identification of a number of cellular proto-oncogenes by virtue of their insertional activation (Varmus, H. “Retroviruses”, Science, 240:1427-1435 (1988)). The possibility that a similar mechanism may cause cancers in patients treated with RVs carrying therapeutic genes intended to treat other pre-existent medical conditions, has posed a recurring ethical problem. Most researchers would agree that the probability of the replication defective RV, such as all those currently used, integrating into or near a cellular gene involved in controlling cell proliferation is vanishingly small. However, it is generally also assumed that the explosive expansion of a population of replication competent retrovirus from a single infection event, will eventually provide enough integration events to make such a phenotypic integration a very real possibility.

Retroviral vector systems are optimized to minimize the chance of replication competent virus being present. However, it has been well documented that recombination events between components of the RV system can lead to the generation of potentially pathogenic replication competent virus and a number of generations of vector systems have been constructed to minimize the risk of recombination (Salmons, B. and Günzburg, W. H., Human Gene Therapy, 4:129-141 (1993)). However, little is known about the finite probability of these events. Since it will never be possible to reduce the risk associated with this or other viral vector systems to zero, an informed risk-benefit decision will always have to be taken. Thus it becomes very important to empirically determine the chance of (Donehower, L. A. et al., J. Virol., 37:226-238, (1981)) insertional disruption or activation of single genes by retrovirus integration and (Kennedy, N. et al., Nature, 295:622-624 (1982)) the risk of generation of replication competent virus by recombination in current generations of packaging cell lines. A detailed examination of the mechanism by which these events occur will also allow the construction of new types of systems designed to limit these events.

A further consideration for practical in vivo gene therapy, both from safety considerations as well as from an efficiency and from a purely practical point of view, is the targeting of RVs. It is clear that therapeutic genes carried by vectors should not be indiscriminately expressed in all tissues and cells, but rather only in the requisite target cell. This is especially important if the genes to be transferred are toxin genes aimed at ablating specific tumour cells. Ablation of other, nontarget cells would obviously be very undesirable. Targeting of the expression of carried therapeutic genes can be achieved by a variety of means.

Retroviral vector systems consist of two components:

1. the retroviral vector itself is a modified retrovirus (vector plasmid) in which the genes encoding for the viral proteins have been replaced by therapeutic genes optionally including marker genes to be transferred to the target cell. Since the replacement of the genes encoding for the viral proteins effectively cripples the virus it must be rescued by the second component in the system which provides the missing viral proteins to the modified retrovirus.

The second component is:

2. a cell line that produces large quantities of the viral proteins, however lacks the ability to produce replication competent virus. This cell line is known as the packaging cell line and consists of a cell line transfected with a second plasmid carrying the genes enabling the modified retroviral vector to be packaged. This plasmid directs the synthesis of the necessary viral proteins required for virion production.

To generate the packaged vector, the vector plasmid is transfected into the packaging cell line. Under these conditions the modified retroviral genome including the inserted therapeutic and optional marker genes is transcribed from the vector plasmid and packaged into the modified retroviral particles (recombinant viral particles). A cell infected with such a recombinant viral particle cannot produce new vector virus since no viral proteins are present in these cells. However, the vector carrying the therapeutic and marker genes is present and these can now be expressed in the infected cell.

Promoter Conversion Vectors

The retroviral genome consists of an RNA molecule with the structure R-U5-gag-pol-env-U3-R (FIG. 6). During the process of reverse transcription, the U5 region is duplicated and placed at the right hand end of the generated DNA molecule, whilst the U3 region is duplicated and placed at the left hand end of the generated DNA molecule (FIG. 6). The resulting structure U3-R-U5 is called LTR (Long Terminal Repeat) and is thus identical and repeated at both ends of the DNA structure or provirus. The U3 region at the left hand end of the provirus harbours the promoter (see below). This promoter drives the synthesis of an RNA transcript initiating at the boundary between the left hand U3 and R regions and terminating at the boundary between the right hand R and U5 region (FIG. 6). This RNA is packaged into retroviral particles and transported into the target cell to be infected. In the target cell the RNA genome is again reverse transcribed as described above.

According to the procon principle a retroviral vector is constructed in which the right hand U3 region is altered (FIG. 7), but the normal left hand U3 structure is maintained (FIG. 7); the vector can be normally transcribed into RNA utilizing the normal retroviral promoter located within the left hand U3 region (FIG. 7). However, the generated RNA will only contain the altered right hand U3 structure. In the infected target cell, after reverse transcription, this altered U3 structure will be placed at both ends of the retroviral structure (FIG. 7).

If the altered region carries a polylinker (see below) instead of the U3 region then any promoter, including those directing tissue specific expression (see below) can be easily inserted. This promoter will then be utilized exclusively in the target cell for expression of linked genes carried by the retroviral vector. Alternatively or additionally DNA segments homologous to one or more cellular sequences can be inserted into the polylinker for the purposes of gene targeting.

In the packaging cell line the expression of the retroviral vector is regulated by the normal unselective retroviral promoter (FIG. 7). However, as soon as the vector enters the target cell promoter conversion occurs, and the therapeutic genes are expressed from a tissue specific promoter of choice introduced into the polylinker (FIG. 7). Not only can virtually any tissue specific promoter be included in the system, providing for the selective targeting of a wide variety of different cell types, but additionally, following the conversion event, the structure and properties of the retroviral vector no longer resembles that of a virus. This, of course, has extremely important consequences from a safety point of view, since ordinary or state of the art retroviral vectors readily undergo genetic recombination with the packaging vector to produce potentially pathogenic viruses. Promoter conversion (Procon) vectors do not resemble retroviruses because they no longer carry U3 retroviral promoters after conversion thus reducing the possibility of genetic recombination.

### SUMMARY OF THE NVENTION

It is an object of the present invention to provide novel usages for the nucleotide and amino acid sequences comprising Naf activity.

It is a further object of the present invention to provide novel usages for the nucleotide and amino acid sequences comprising Sag activity.

It is also a further object of the present invention to provide novel vectors useful for gene therapy of viral infections.

It is still a further object of the present invention to provide novel vectors useful for gene therapy of diseases associated with B cells.

According to one aspect of the present invention there is provided a novel usage of a nucleotide sequence or amino acid sequence of a derivative thereof comprising Naf activity for repressing the expression of viral promoters, e.g., for the treatment of viral infections.

In another aspect the invention provides a novel recombinant DNA vector for introducing into an eucaryotic cell DNA for repressing the expression of heterologous viral promoters, the vector comprising, in operable linkage, a) the DNA of or corresponding to at least a portion of a vector, which portion is capable of infecting and directing the expression in the target cells; and b) one or more coding sequences wherein at least one sequence encodes for a peptide (protein) with Naf activity or a derivative thereof. Optionally, the recombinant vector of the present invention can include at least one sequence encoding a therapeutic and/or non-therapeutic peptide (protein). For example, the peptide (protein) can be β-galactosidase, neomycin, alcohol dehydrogenase, puromycin, hypoxanthine phosphoribosyl transferase (HPRT), hygromycin, secreted alkaline phosphatase, Herpes Simplex Virus thymidine kinase, cytosine deaminase, guanine phosphoribosyl transferase (gpt), cytochrome P 450, cell cycle regulatory genes which codes for proteins including P.T.O. or SDI, tumor supressor gene which codes for proteins including p53, antiproliferation genes which codes for proteins including melittin and cecropin, or genes which codes for cytokines such as IL-2.

Said vector is selected from the group of viral and plasmid vectors. In particular said viral vector is selected from the group of RNA and DNA viruses. Said plasmid vector is preferably selected from the group of eucaryotic expression vectors and wherein said RNA virus vector is selected from retrovirus vectors. Said DNA virus is preferably selected from the group of adenoviruses, adenovirus associated viruses and herpes viruses; and wherein said retroviral vector is preferably selected from the group of procon vectors. In a preferred embodiment the retroviral genome is replication-defective.

In one embodiment the present invention uses the principle of promoter conversion typical for retroviruses.

The procon vector includes preferably, in operable linkage, a 5′LTR region; one or more of said coding sequences wherein at least one sequence encodes for a peptide with Naf activity or a derivative thereof for repressing the expression of heterologous viral promoters; and a 3′LTR region; said 5′LTR region comprising the structure U3-R-U5 and said 3′LTR region comprising a completely or partially deleted U3 region wherein said deleted U3 region is replaced by a polylinker sequence, followed by the R and U5 region to undergo promoter conversion.

In a further preferred embodiment, the retrovirus vector includes, in operable linkage, a 5′LTR region and a 3′LTR region, said 5′LTR region comprising the structure U3-R-U5 and said 3′LTR region comprising a completely or partially deleted U3 region wherein said deleted U3 region is replaced by one or more of said coding sequences wherein at least one sequence encodes for a peptide with Naf activity expressed from either the viral or a heterologous promoter for repressing the expression of heterologous viral promoters followed by the R and U5 region.

With reference to the procon vectors, said polylinker sequence carries at least one unique restriction site and contains preferably at least one insertion of a heterologous DNA fragment. Said heterologous DNA fragment is preferably selected from regulatory elements and promoters, preferably being target cell specific in their expression.

For a complete disclosure of the procon vectors, the content of the Danish application DK1017/94, filed on Sep. 2, 1994 is completely included within the present application or incorporated herein by reference.

The recombinant DNA vectors provided by the present invention may preferably be used to treat viral infections by repressing viral promoters.

The recombinant DNA vectors provided in the present invention may be preferably used to repress heterologous viral promoters selected from HIV or MLV promoters.

In a further aspect the invention provides a novel usage of a nucleotide sequence or amino acid sequence or a derivative thereof comprising Sag activity in the gene therapy of disorders associated with B or T cells.

Recombinant vectors comprising nucleotide sequence encoding a peptide with sag-activity or a derivative thereof are particularly useful, if antigen presenting cells are the target cells for the recombinant vector. After the introduction of the vectors according to the present invention into antigen presenting cells, such as B-lymphocytes, Sag stimulates the proliferation of whole classes of T-cells bearing the cognate Vβ chain as part of their T-cell receptor. This T-cell activation results in the proliferation of B-cells in the vicinity, including those who were infected with the vector. In summary, the infection of antigen presenting cells with the recombinant vectors expressing peptides with sag-activity leads to an increase in the number of cells containing the recombinant vector. Therefore, the vectors according to the present invention are particularly suitable for gene therapy protocols where it is often a problem to obtain a sufficient number of cells harboring the recombinant vector.

In the context of the present invention the term “sag activity” refers to the known activity of a superantigen to result in the stimulation and growth, followed by deletion or anergy, of reactive cells (Gunzburg et al., Nature, 364:154-158 (1993); Choi, Y., et al., Nature, 350:203-207 (1991); Acha-Orbea, H., et al., Nature, 350:207-211 (1991)). The superantigen expression can be determined and quantified in a “mixed lymphocyte reaction” (Wintersperger et al., BioTechniques, 16:882-884 (1994)). A surplus of superantigen presenting lymphoma cells is co-cultivated for 4 days with freshly prepared T-lymphocytes. Depending on the origin of the superantigen being presented, one or more specific T-cell class bearing responding β-chains (Vβ) as parts of their T-cell receptors are stimulated to proliferate. These cells can further be labeled with FITC-conjugated Vβ-subclass specific antibodies and subsequently analyzed by FACS.

In the context of the present invention “derivatives of the peptide with sag activity” are peptides having the activity of Sag as defined above but differing in the amino acid sequence of known Sag-peptides in one or more positions. Typical examples of derivatives of peptides with Sag-activity are: (1) sag-peptides with one or more conservative amino acid substitutions; (2) sag-peptides with one or more amino acid deletions; (3) sag-peptides with one or more amino acid insertions, compared to known sag sequences; or (4) any functional combination hereof. Typical examples of non-random variations within the sag sequence are shown by Brandt-Carlson et al., Virol., 193:171-185 (1993)).

In a preferred embodiment, a recombinant DNA vector for introducing into a B or T cell DNA for gene therapy of disorders associated with B or T cells is provided, comprising, in operable linkage,

a) the DNA of or corresponding to at least a portion of a vector, which portion is capable of infecting and directing the expression in the B or T cells; and

b) one or more coding sequences wherein at least one sequence encodes for a peptide with Sag activity or a derivative thereof and at least one sequence encodes for a therapeutic peptide or protein.

Said vector is selected from the group of viral and plasmid vectors. In particular said viral vector is selected from the group of RNA and DNA viruses. Said plasmid vector is preferably selected from the group of eucaryotic expression vectors and wherein said RNA virus vector is selected from retrovirus vectors. Said DNA virus is preferably selected from the group of adenoviruses, adenovirus associated viruses and herpes viruses; and wherein said retroviral vector is preferably selected from the group of procon vectors. In a preferred embodiment the retroviral genome is replication-defective.

In a preferred embodiment said procon vector includes, in operable linkage, a 5′LTR region; one or more of said coding sequences wherein at least one sequence encodes for a peptide with Sag activity or a derivative thereof and at least one sequence encodes for a therapeutic peptide; and a 3′LTR region; said 5′LTR region comprising the structure U3-R-U5 and said 3′LTR region comprising a completely or partially deleted U3 region wherein said deleted U3 region is replaced by a polylinker sequence, followed by the R and U5 region to undergo promoter conversion.

According to a further preferred embodiment a retrovirus vector is used which includes, in operable linkage, a 5′LTR region and a 3′LTR region, said 5′LTR region comprising the structure U3-R-U5 and said 3′LTR region comprising a completely or partially deleted U3 region wherein said deleted U3 region is replaced by one or more of said coding sequences wherein at least one sequence encodes for a peptide with Sag activity or a derivative thereof and at least one sequence encodes for a therapeutic peptide (protein) expressed from either the viral or a heterologous promoter, followed by the R and U5 region.

Gene expression is regulated by promoters. In the absence of promoter function a gene will not be expressed. The normal MLV retroviral promoter is fairly unselective in that it is active in most cell types. However, a number of promoters exist that show activity only in very specific cell types. Such tissue-specific promoters will be the ideal candidates for the regulation of gene expression in retroviral vectors, limiting expression of the therapeutic genes to specific target cells.

The target cell specific regulatory elements and promoters are preferably, but not limited, selected from one or more elements of the group consisting of HIV, Whey Acidic Protein (WAP), Mouse Mammary Tumour Virus (MMTV), β-lactoglobulin and casein specific regulatory elements and promoters, which may be used to target human mammary tumours, pancreas specific regulatory elements and promoters including carbonic anhydrase II and β-glucokinase regulatory elements and promoters, lymphocyte specific regulatory elements and promoters including immunoglobulin and MMTV lymphocytic specific regulatory elements and promoters and MMTV specific regulatory elements and promoters conferring responsiveness to glucocorticoid hormones or directing expression to the mammary gland, T-cell specific regulatory elements and promoters such as T-cell receptor gene and CD4 receptor promoter and B-cell specific regulatory elements and promoters such as immunoglobulin promoter or mb1. Said regulatory elements and promoters regulate preferably the expression of at least one of the coding sequences of said retroviral vector.

The LTR regions are preferably, but not limited, selected from at least one element of the group consisting of LTRs of Murine Leukaemia Virus (MLV), Mouse Mammary Tumour Virus (MMTV), Murine Sarcoma Virus (MSV), Simian Immunodeficiency Virus (SIV), Human Immunodeficiency Virus (HIV), Human T-cell Leukaemia Virus (HTLV), Feline Immunodeficiency Virus (FIV), Feline Leukaemia Virus (FELV), Bovine Leukaemia Virus (BLV) and Mason-Pfizer-Monkey virus (MPMV).

The Naf or Sag encoding sequences of the present invention will be placed under the transcriptional control of, for instance, the HIV promoter or a minimal promoter placed under the regulation of the HIV tat responsible element (TAR) to target HIV infected cells. Targeting will be achieved because the HIV promoter is dependent upon the presence of Tat, an HIV encoded autoregulatory protein (Haseltine, W. A., FASEB J., 5:2349-2360 (1991)).

Thus only cells infected with HIV and therefore expressing Tat will be able to produce the Naf or Sag peptide encoded by the vector. Alternatively, the Naf or Sag peptide could be expressed from T cell specific promoters such as that from the CD4 or T cell receptor gene. In order to target tumour cells, promoters from genes known to be overexpressed in these cells (for example c-myc, c-fos) may be used.

The Naf or Sag encoding sequences of the present invention may be placed also under the transcriptional control of other promoters known in the art. Examples for such promoters are of the group of SV40, cytomegalovirus, Rous sarcoma virus, β-actin, HIV-LTR, MMTV-LTR, target cell specific promoters, B or T cell specific promoters and tumour specific promoters.

In one embodiment of the invention the Naf or Sag peptide is expressed from MMTV promoters such as the ^{MMTV}P2 promoter (Günzburg, W. H., et. al., Nature, 364:154-158 (1993)).

The retroviral vector is in one embodiment of the invention a BAG vector (Price, J. D., et. al., Proc. Natl. Acad. Sci. USA, 84:156-160 (1987)), but includes also other retroviral vectors.

According to a preferred embodiment of the invention at least one retroviral sequence encoding for a retroviral protein involved in integration of retroviruses is altered or at least partially deleted.

The vector preferably contains DNA fragments homologous to one or more cellular sequences. The regulatory elements and promoters are preferably regulatable by transacting molecules.

In a further embodiment of the invention a retroviral vector system is provided comprising a retroviral vector as described above as a first component and a packaging cell line harbouring at least one retroviral or recombinant retroviral construct coding for proteins required for said retroviral vector to be packaged.

The packaging cell line harbours retroviral or recombinant retroviral constructs coding for those retroviral proteins which are not encoded in said retroviral vector. The packaging cell line is preferably selected from an element of the group consisting of ψ2, ψ-Crip, ψ-AM, GP+E−86, PA317 and GP+envAM−12.

After replicating the retroviral vector of the invention as described above in a retroviral vector system as described above, a retroviral provirus is provided wherein U3 or said polylinker and any sequences inserted in said polylinker in the 3′LTR become duplicated during the process of reverse transcription in the infected target cell and appear in the 5′LTR as well as in the 3′LTR of the resulting provirus, and the U5 of 5′LTR become duplicated during reverse transcription and appear at the 3′LTR as well as in the 3′LTR of the resulting provirus.

According to the invention the term “polylinker” is used for a short stretch of artificially synthesized DNA which carries a number of unique restriction sites allowing the easy insertion of any promoter or DNA segment. The term “heterologous” is used for any combination of DNA sequences that is not normally found intimately associated in nature. The retroviral vector of the invention refers to a DNA sequence retroviral vector on the DNA sequence level.

The invention includes, however, also MRNA of a retroviral provirus according to the invention and any RNA resulting from a retroviral vector according to the invention and cDNAs thereof.

A further embodiment of the invention provides non-therapeutical or therapeutical method for introducing Naf or Sag sequences into human or animal cells in vitro and in vivo comprising transfecting a packaging cell line of a retroviral vector system according to the invention with a retroviral vector according to the invention and infecting a target cell population with recombinant retroviruses produced by the packaging cell line.

The retroviral vector, the retroviral vector system and the retroviral provirus as well as RNA thereof may be used for producing a pharmaceutical composition for somatic gene therapy in mammals including humans. Furthermore, they are used for targeted integration in homologous cellular sequences.

The retroviral promoter structure is termed LTR. LTR's carry signals that allow them to jump in and out of the genome of the target cell. Such jumping transposable elements can also contribute to pathogenic changes. Retroviral vectors vectors can carry modified LTRs that no longer carry the signals required for jumping. Again this increases the potential safety of these vector systems.

Further objects, features and advantages will be apparent from the following description of preferred embodiments of the invention.

### BRIEF DESCRIPTION OF THE DRAWINGS

FIG. 1 is a schematic of the MMTV Naf/Sag expression plasmid pORFexp. The pORFexp plasmid was derived from pGR102 (upper construct), which contains a complete biologically active MMTV provirus (Salmons, B. et. al., Virology, 144:101-114 (1985)), by digestion with NcoI(N) to remove the part of the gag as well as the pol and env regions followed by religation. Indicated are the U3, R and U5 regions of the LTR as well as the gag, pol and env genes and the two transcriptional starts (arrowed) within the 5′LTR. The open reading frame is indicated by the shaded box in the U3 region. Restriction enzyme cleavage sites for HpaI(H), PvuII(P), ScaI(Sc), BglII(B) and StuI(St) used in the construction of the pORFexp derived plasmids pORFexp o/c (No. 2 in FIG. 2A), pdelU3 (No. 5 in FIG. 2A), pdelRU5 (No. 6 in FIG. 2A), pdelgag (No. 4 in FIG. 2A) and pSVorfexp (No. 3 in FIG. 2A), (FIG. 3) are also indicated. The splice donor (SD) at the 5′ end of the gag gene and a second splice donor (SD) are indicated as is the splice acceptor (SA) for ORF. PCR primers +1702 and −3228 used to demonstrate the transcript that utilized the second splice donor in the gag gene are shown as arrows below pORFexp. Also shown is part of the determined sequence (SEQ ID NO: 1) of the PCR product from the splice junction region which confirms the use of the second splice donor in the gag and the splice acceptor in the ORF.

FIG. 2A is a schematic of the following expression constructs derived from pORFexp (construct 1) construct No. 2 pORFexp o/c which carries a premature termination codon within the open reading frame carried within the 3′MMTV LTR (indicated by X); construct No. 3 pSVorfexp in which ORF products are transcribed directly from a SV40 promoter; construct No. 4 pdelgag in which the gag region has been removed; construct No. 5 pdelU3 carrying only the classic MMTV promoter; and construct No. 6 pdelRU5 carrying the only novel promoter.

FIG. 2B is a graph showing the mean value of 3 independent experiments in which luciferase activity from the indicator constructs pRSVluc (solid bars), pMtv2luc (dotted bars) and pMtv9luc (striped bars) was measured after transient transfection into either CK cells or cell clones that have stably acquired the indicated expression constructs. At least two individual clones carrying each construct were tested in the luciferase assay to rule out clonal variation effects.

FIG. 2C is a graph showing the ability of each of the expression constructs to direct superantigen activity after electroporation into A20 cells in a mixed lymphocyte reaction. The 3′LTR of these constructs is derived from the Mtv-2 provirus, the superantigen of which stimulates specifically the growth of Vβ14 bearing T cells (Günzburg, W. H. et al., Nature, 364:154-158 (1993), Acha-Orbea, H., et al., Nature, 350:207-211 (1991) and Hornsby, P. et. al., Bio Techniques, 12:244-251 (1992)). The percentage of Vβ14 bearing T cells (solid bars) in the total population of T cells (CD3+cells) is shown as is the percentage of nonresponding, Vβ8 bearing, T cells (open bars). An increase in Vβ14 bearing T cells and a concomitant decrease in Vβ8 bearing T cells is indicative of Mtv-2 superantigen activity.

FIG. 3 is a schematic of indicator constructs used to measure Naf medicated downregulation. All of the constructs carry a promoterless luciferase gene coupled to the indicated heterologous promoters and transcription termination sequences from SV40 (SV40pA). Relevant restriction enzyme sites are indicated: BamHI(Ba), BglII (B), HindIII(H), SalI(Sa), SpeI(Sp) and EcoRI (E).

FIG. 4 is a bar graph showing down regulation of luciferase expression from the HSVtk (pT109luc), RSV (pRSVluc), MMTV (pMtvluc), HIV (pHIVluc), MLV (pMLVluc) but not the β-actin (pβ-actinluc) promoter by Naf. CK, COE3 and COE12 cells were transiently transfected with the indicated plasmids and cell extracts prepared 48 hours post transfection. Equivalent amounts of protein were used for luciferase assay as described (Hornsby, P. et. al., Bio Techniques, 12:244-251 (1992)). The luciferase activity of each construct in CK cells was taken as 100% (dotted line) and the mean and range of 3 independent experiments is shown for COE3 (dotted boxes) and CEO12 (open boxes).

FIG. 5 is a schematic drawing showing a retroviral vector according to one embodiment of the invention wherein at least part of the U3 region is replaced by a Sag coding sequence or a derivative thereof.

FIG. 6 is a schematic of reverse transcription of a retroviral genome.

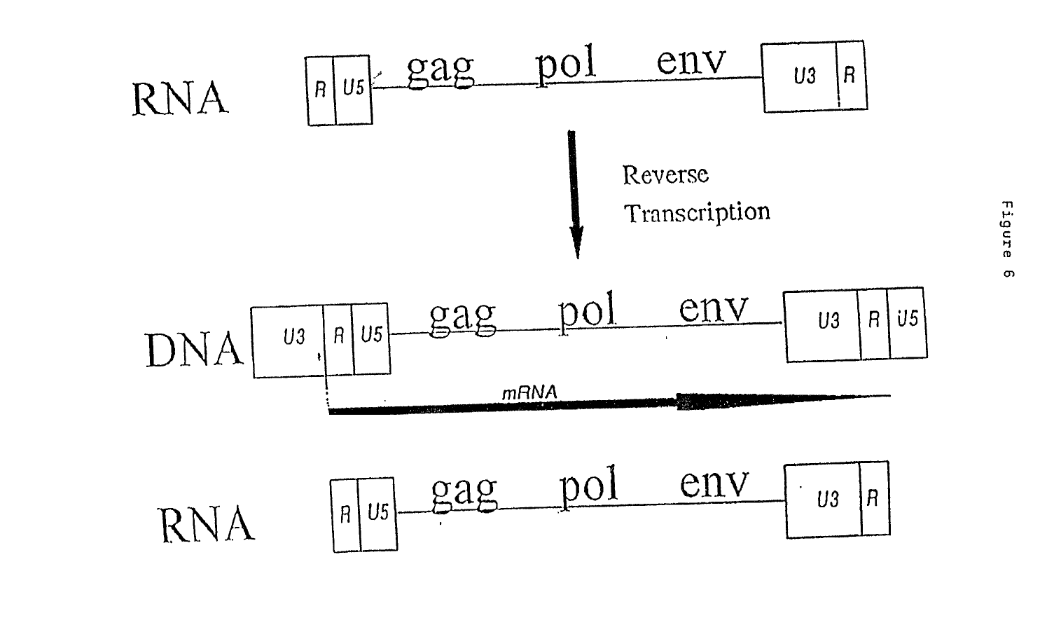

FIG. 7 is a schematic of the procon principle wherein a U3 minus BAG-vector is constructed.

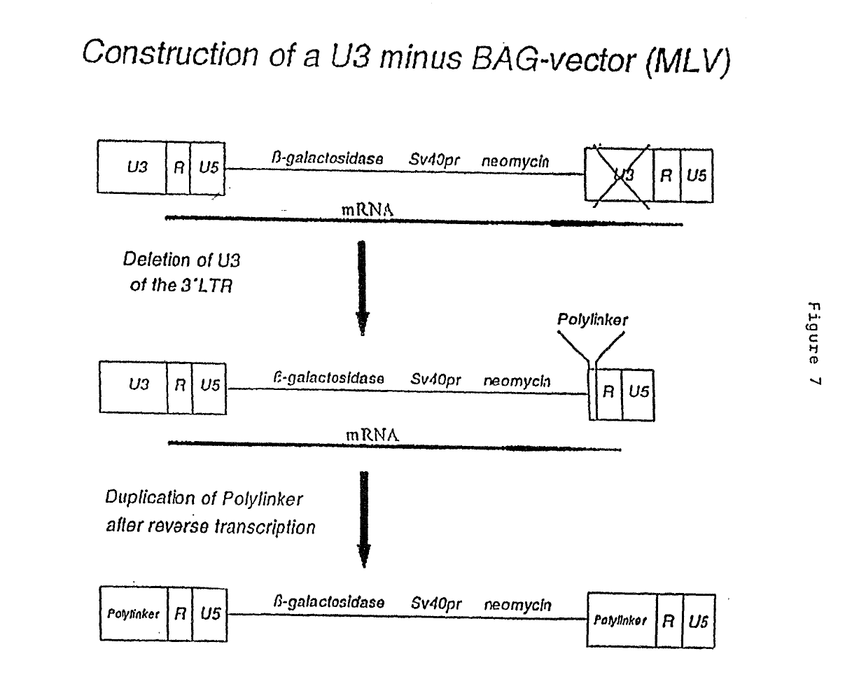

### DETAILED DESCRIPTION OF THE INVENTION

The invention refers to novel recombinant vectors useful for gene therapy of viral infections and of diseases associated with B and T cells. The present invention relates, furthermore, to novel usages of the two products of the open reading frame of mouse mammary tumour virus.

The superantigen activity, encoded by the MMTV ORF appears to be crucial for the transfer of MMTV from the gastric tract where the virus is delivered in the milk, to the mammary gland. One of the first cells to become infected by the ingested MMTV are B cells. These infected B cells express the virally encoded Sag, possibly from a recently described, second viral promoter (Günzburg, W. H. et al., Nature, 364:154-158 (1993)) on the cell surface. This presentation of Sag protein in combination with MHC class II molecules results in the stimulation of specific classes of T cells according to the V13 chain that they carry as part of the T cell receptor. Such activated T cells are stimulated to produce cytokines which then cause the local proliferation of B cells, which includes the original MMTV infected B cells (reviewed in (Acha-Orbea, H., et al., Nature, 350:207-211(1991)). Thus the initial few infected B cells are amplified and form a reservoir which eventually passes the virus on to the mammary gland.

It was surprisingly shown that the expression of the MMTV encoded superantigen from a retroviral or other type of vector system carrying an additional, B or T cell specific therapeutic gene permits the expansion of B or T cells bearing the introduced genes. Thus superantigen may be used to enrich in vivo genetically modified B cells by using a naturally occurring amplification mechanism. This increases the efficiency of gene transfer to B or T cells.

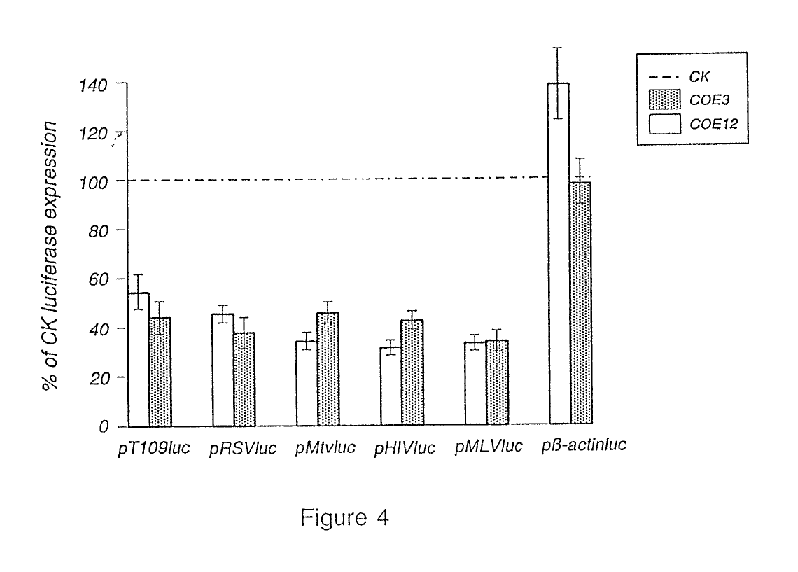

As shown herein, Naf acts in trans to downregulate expression from MMTV by reducing the rate of transcription (Salmons, B., et al., J. Virol., 64:6355-6359, (1990)). Surprisingly, as also shown herein, the effects of Naf are not limited to MMTV; Naf represses also the expression of a number of retroviral promoters including those of Human Immunodeficiency virus (HIV) and Murine Leukemia Virus (MLV). This provides evidence that Naf induced down regulation is mediated by a common transcription factor (FIG. 4). The ability of Naf to negatively regulate retroviral promoters enables use of Naf in gene therapy towards the treatment of viral infections, in particular of HIV infections. One such strategy involves the delivery of a Naf expression system (for example in a retroviral or other gene transfer vector systems) specifically to HIV infected cells from AIDS patients, in order to inhibit virus expression and replication. Further, Naf may also be useful as a means for controlling the expression from MLV based retroviral vectors in other gene therapy protocols.

The following examples will illustrate the invention further. These examples are however in no way intended to limit the scope of the present invention as obvious modifications will be apparent, and still other modifications and substitutions will be apparent to anyone skilled in the art.

The recombinant DNA methods employed in practicing the present invention are standard procedures, well known to those skilled in the art, and described in detail, for example, in Molecular Cloning, Sambrook, et. al., Cold Spring Harbor Laboratory, (1989) and B. Perbal, A Practical Guide to Molecular Cloning, John Wiley & Sons (1984).

Materials and Methods

Plasmids

a) Expression Constructs

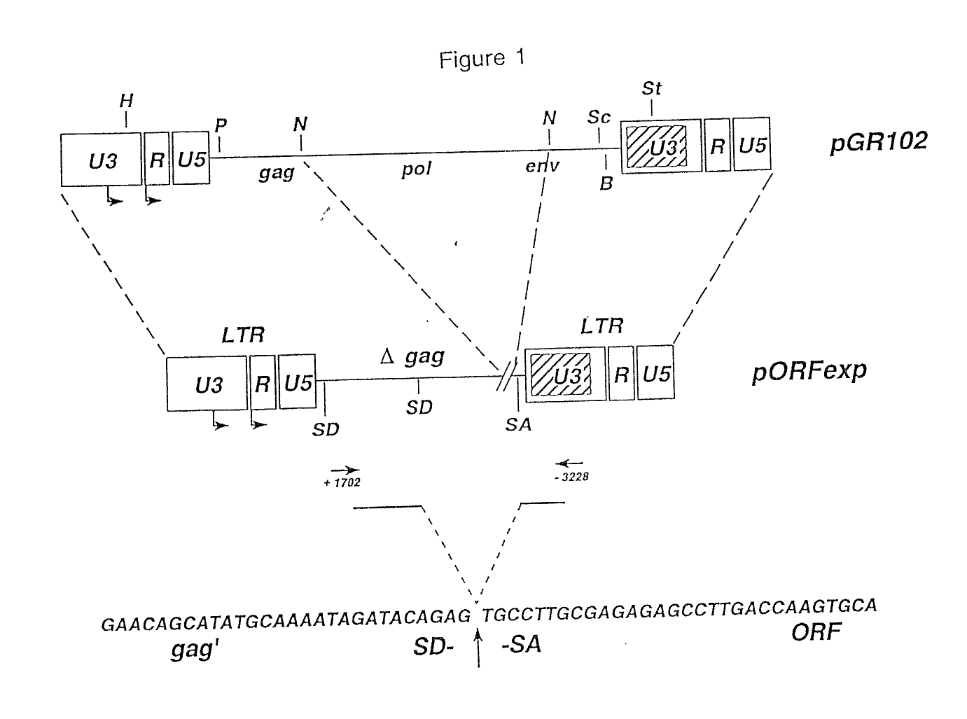

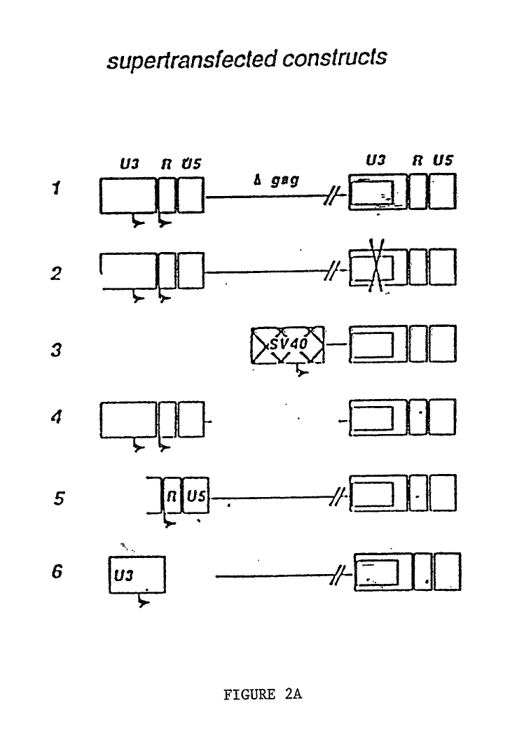

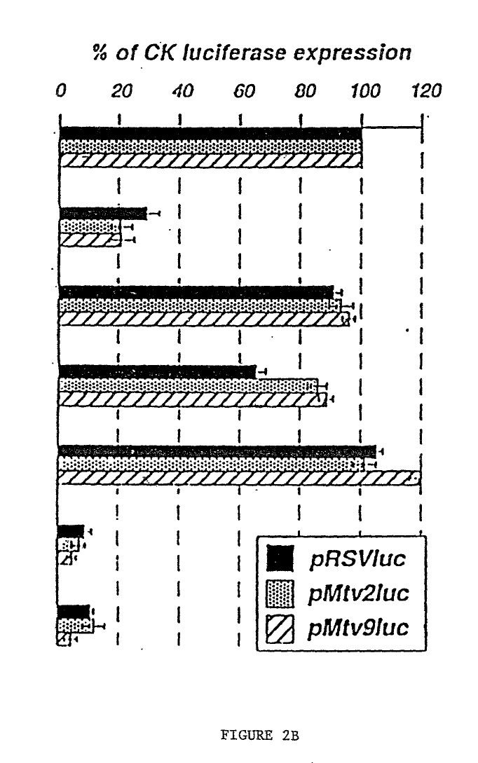

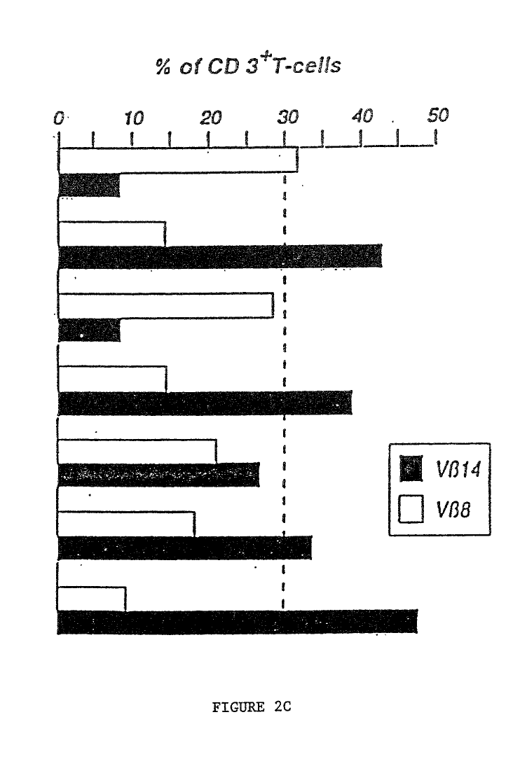

The pORFexp expression plasmid was constructed by digesting pGR102 (Salmons, B. et. al., Virology, 144:101-114 (1985)) with NcoI to remove part of the gag, pol and env sequences followed by religation (FIG. 2A). A series of plasmids were derived from pORFexp (FIG. 1); pORFexp o/c (construct 2; FIG. 2A) carries a ClaI linker at the StuI site in the 3′LTR (FIG. 1) creating a premature stop codon leading to a truncation of the predicted ORF product (Salmons, B., et al., J. Virol., 64:6355-6359, (1990)); pdelgag (construct 4; FIG. 2A) by digestion of pORFexp with PvuII and ScaI (FIG. 1) followed by religation; pdelU3 (construct 5; FIG. 2A) by digestion of pORFexp with EcoRV (in the 5′ vector sequences) and HpaI (FIG. 1) to remove most of the U3 region including the novel upstream promoter in the 5′LTR, followed by religation; pdelRU5 (construct 6; FIG. 2A) by the removal of a HpaI/PvuII fragment from pORFexp (FIG. 1), which deletes a small part of the U3, the R and U5 regions of the 5′LTR thereby removing the classic promoter but leaving the novel promoter intact; pSVorfexp (construct 3; FIG. 2A) by ligation of the BglII/XmnI SV40 promoter containing fragment of pSV2neo (Southern, P. J. and Berg, P., J. Mol. App. Gen., 1:327-341 (1982)) to a BglII/XmnI 3′LTR containing fragment of pORFexp (FIG. 1).

b) Indicator Constructs

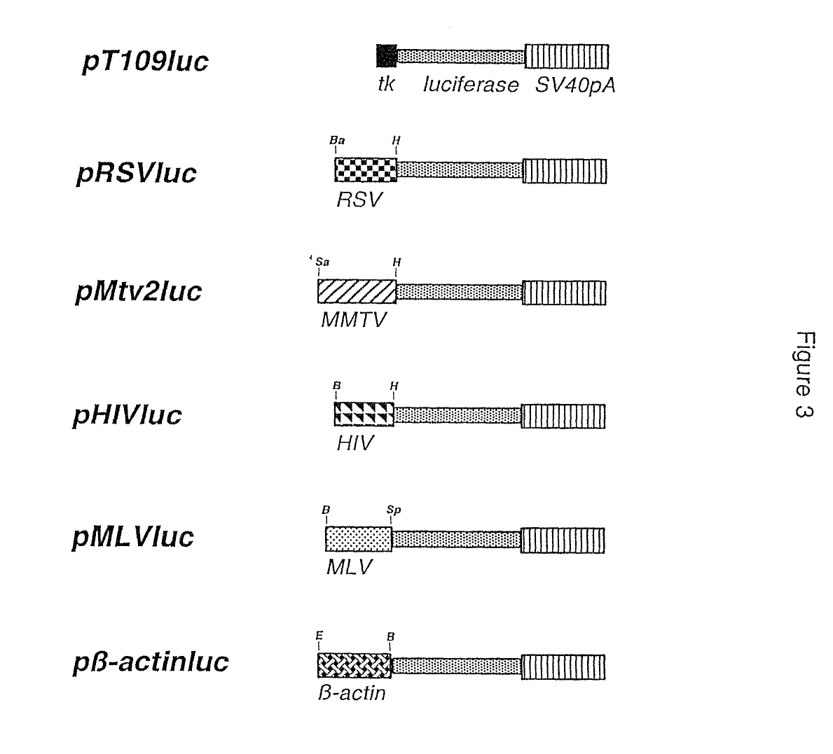

Expression plasmids carrying the luciferase gene under the transcriptional control of a number of heterologous promoters (FIG. 3) were used to determine whether these promoters are Naf responsive: pT109luc (Nordeen, S. K., Biotechniques, 6:454-458 (1988)) carries a 132 bp BamHI-BglII fragment of the herpes simplex virus thymidine kinase promoter; pRSVluc carries a 550 bp BamHI-HindIII fragment comprising the promoter of Rous Sarcoma Virus (RSV) contained in the LTR; pMtv2luc was constructed in the following way. The HpaII site of a BglII-HpaII DNA fragment containing the complete MMTV LTR from an exogenous milk borne virus was converted into a BamHI site and the resultant fragment cloned into the BamHI site of pUC18. A SalI-HindIII fragment of the resulting plasmid was then cloned into the plasmid pLUC1, which carries a promoterless luciferase gene (Günzburg, W. H. et al., Nature, 364:154-158 (1993)). pMtv9luc carries a 1200 bp PstI-EcoRi DNA fragment containing the entire LTR of the endogenous Mtv-9 provirus linked to the luciferase gene (Lund, F. E. and Corley, R. B., J. Exp. Med., 174:1439-1450 (1991)); pHIVluc was constructed by cloning a 560 bp BglII-HindIII DNA fragment of the human immunodeficiency virus (HIV-1) LTR lacking the NRE into the same sites of pLUC1; pMLVluc carries the complete murine leukemia virus (MLV) LTR within a 704 bp BglII-SpeI DNA fragment from a recombinant polymerase chain reaction (PCR) (using the primers 5′CGCAGATCTTAGCTTAAGTAACGCCATT3′ (SEQ ID NO: 2) and 5′CGCACTAGTTCCGCCAGATACAGAG3′ (SEQ ID NO: 3)) ligated into the same sites in pLUC1; p13-actinluc (Langer, S. J. and Ostrowski, M. C., Mol. Cell. Biol., 8:3872-3881 (1988)) carries a EcoRI-BamHI 13-actin promoter containing DNA fragment from the plasmid pHβAPr-1-neo coupled to the luciferase gene.

RT-PCR Analysis

RNA was isolated from transfected cells, reverse transcribed into DNA and used in PCR reactions as previously described (Günzburg, W. H. et. al., Nature, 364:154-158 (1993). The primers +1702 (5′GAGGTACGCAGCGGAACA3′) (SEQ ID NO: 4) and −3228 (5′TGATGGGCTCATCCGTTT3′) (SEQ ID NO: 5), specific for the gag and ORF region (FIG. 1) were used for PCR reactions and resultant products were sequenced using the same primers on an ABI-373A automated DNA sequence (Applied Biosystems).

Cell Culture

A20 cells, derived from a B-cell lymphoma of a Balb/c mouse (2G), were cultured in RPMI medium containing 5% fetal bovine serum, L-glutamine and mercaptoethanol. CK cells, derived from the feline kidney cell line CFRK (Crandell, R. A. et. al., In vitro 9, 1:76-185 (1973), and GR mouse mammary carcinoma cells, productively infected with MMTV (Salmons, B. et. al., Virology, 144:101-114 (1985)) were maintained in Dulbecco's MEM containing 10% fetal bovine serum.

Transfection

CK cells, seeded at a density of 5×10⁵ cells per 10 cm dish, were co-transfected with 5 μg of pORFexp and 0.5 μg pRSVneo using the Cellphect kit (Pharmacia) according to the manufacturers' instructions. Stably transfected G418 resistant (400 μg/ml) cell clones were isolated two weeks post transfection. Two of these clones, COE3 and COE12, were shown to carry and express the pORFexp construct. Each of the various pORFexp derived expression constructs were also co-transfected into CK cells at a 20:1 ratio with pX343, a plasmid conferring hygromycin resistance. Stably transfected hygromycin resistant cell clones or populations were isolated 15-17 days after transfection and selection in 100 μg/ml hygromycin. Transfected clones were used for supertransfection with 5 μg of the luciferase carrying constructs.

Luciferase Assay

Cell extracts were prepared for luciferase assays 48 hours post transfection as described previously (Hornsby, P. et. al., Bio Techniques, 12:244-251 (1992)). The protein concentration of the samples was determined by the Bradford assay technique (Bio-Rad, Protein Assay) and 100 ng of protein used for the luciferase assay as described previously (Hornsby, P. et. al., Bio Techniques, 12:244-251 (1992)) using a Berthold AutoLumat LB953.

Superantigen Assay

1×10⁷ A20 cells were resuspended in RPMI containing 20 μg of plasmid in a 0.4 cm cuvette and pulsed with 300V 960 μF (Bio-Rad Gene Pulser) as described previously (Wintersperger, S. et. al., BioTechniques, 16:882-886 (1994)). Twenty hours later, the cells were irradiated (3000 rad) to inhibit growth and 1×10⁷ Cocultured with 2×10⁶ primary T cells freshly isolated from popliteal lymph nodes of Balb/c mice. Four days later, T cells were stained with R-phycoerythrin labelled anti-CD3mAb and either FITC conjugated antiV8 or antiVβ14 mAb and analyzed by FACS (Elite, Coulter Inc.) to determine the percentage of V8 and Vβ14 bearing T cells.

S1 ANALYSIS

Total RNA (40 μg) isolated from CK cells, CK cells transfected with pdelgag, pORFexp o/c or pORFexp or GR cells was hybridized to a BstEII probe as previously described (Günzburg, W. H. et al., Nature, 364:154-158 (1993)). Transcripts initiating at the MMTV promoter protect a fragment of 110nt. The protected fragments were densitometrically evaluated using a Fuji Phosphoimager and the intensity of the 110nt fragment was corrected using the loading control to ensure equal amounts of counts were applied to each lane.

Establishment of Naf Expressing Clones

Previous studies implicated both gag and ORF sequences as encoding Naf (Salmons, B., et al., J. Virol., 64:6355-6359, (1990)). To verify this data, a plasmid, pORFexp, was constructed which carries putative Naf encoding sequences (FIG. 1). Naf mediated transcriptional downregulation was observed upon transfection of pORFexp into RMC2h assay cells (Salmons, B., et al., J. Virol., 64:6355-6359, (1990)). In order to facilitate the detection of potential Naf specific transcripts as well as to further characterize Naf activity, the pORFexp construct was transfected into CK cells, one of the few cultured cell lines that are permissive for MMTV (Salmons, B. et. al., Virology, 144:101-114 (1985); Crandell, R. A. et. al., In vitro 9, 1:76-185 (1973)). A number of resultant cell clones, including COE3 and COE12 (see below), were shown to carry the pORFexp construct in a contiguous form. Transcripts expressed in the pORFexp clones were examined by Northern blot as well as by RT-PCR (FIG. 1). In addition to the previously described MMTV splice donor at the 5′ end of the gag gene (FIG. 1), a novel splice donor within the gag gene was identified. Transcripts using this splice donor also use the previously described splice acceptor for ORF (FIG. 1). A second promoter has recently been described within the U3 region of the MMTV LTR (Günzburg, W. H. et al., Nature, 364:154-158 (1993)). Transcripts initiating at this promoter and utilizing the novel splice donor within the gag gene generate mRNAs of 2.5 kb. ORF specific transcripts of a similar size have been previously reported (Lund, F. E. and Corley, R. B., J. Exp. Med., 174:1439-1450 (1991); Held, W. et. al., J. Exp. Med., 177:359-366 (1993); Lund, F. E. et. al., J. Immunol., 150:78-86 (1993)). The two pORFexp transfected cell clones COE3 and COE12 were further analyzed for functional Naf activity.

Naf Down Regulates Heterologous Viral Promoters

Naf was originally demonstrated to downregulate expression from an MMTV provirus in which the 5′LTR had been replaced by that of Rous sarcoma virus (RSV) (Salmons, B. et. al., J. Virology, 64:6355-6359 (1990)). Thus it was not known whether Naf induced downregulation was mediated by sequences in the RSV promoter or in the linked MMTV provirus. To resolve this issue two constructs in which either the MMTV or RSV LTR is linked to a promoterless luciferase gene (FIG. 3) were transfected into both COE clones as well as into CK cells. The luciferase activity from each construct (pRSVluc and Mtvluc) in two COE clones was around 40% of that observed in CK cells (FIG. 4), whereas luciferase activity from a control β-actin promoter was not reduced.

Surprisingly, it could be demonstrated that both COE clones express functional Naf and that both retroviral promoters are Naf responsive and that Naf downregulates expression from other heterologous retroviral promoters. This could be verified for the promoters carried within the HIV and MLV LTRs (FIG. 4). Surprisingly, the HSVtk promoter was also Naf responsive. Clearly, the downregulatory effects are not due to clonal variation since the extent of luciferase downregulation from each construct was similar in both COE clones. Further, the finding that the β-actin-luciferase construct was not downregulated strongly demonstrates that this is not a nonspecific property of the COE clones. The observation that Naf represses transcription from heterologous promoters as well as from the MMTV LTR provides evidence that Naf acts indirectly via an as yet unidentified common transcription factor.

### Example for the Construction of a Sag Carrying Therapeutic RNA Virus Vector

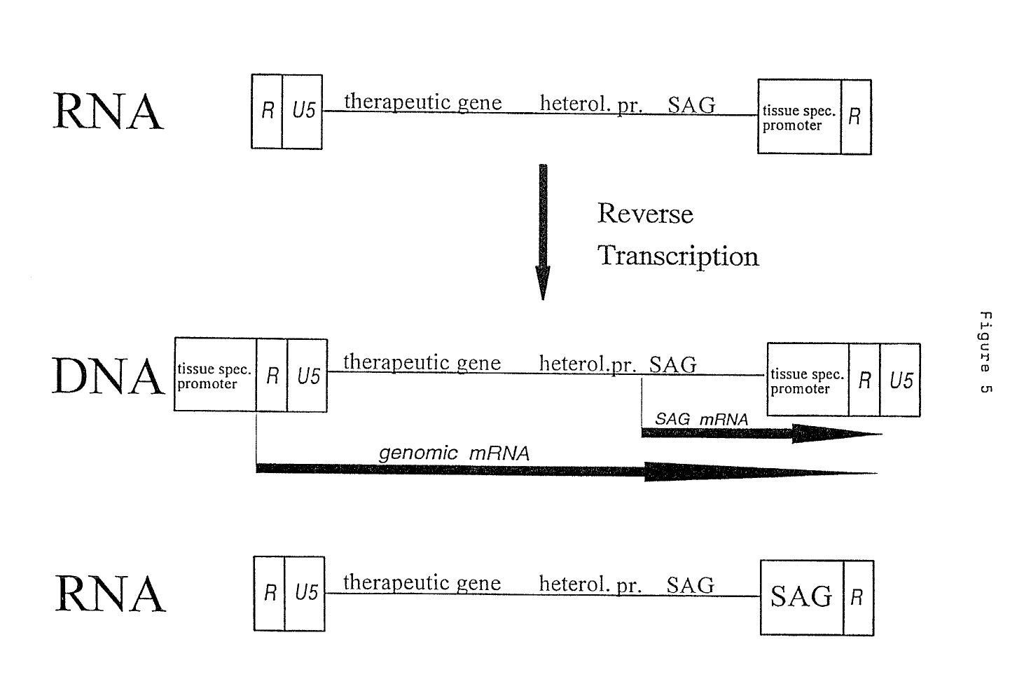

The superantigen encoding sequences are inserted into the retroviral vector either under the transcriptional control of the retroviral promoter or a heterologous promoter. The Sag can be inserted in place of the retroviral structural genes as shown in the accompanying FIG. 5 or in the U3 region of the left hand long terminal repeat (LTR). A procon vector carrying Sag is introduced into a packaging cell line, recombinant virus is produced and used to infect the target cells. Upon infection, the viral genomic RNA is reverse transcribed into a double stranded DNA form, which results is the placement of the Sag sequences in both LTRs, and the DNA is then integrated in the host cell genome where it is expressed like any other cellular gene. A therapeutic gene may in addition to the Sag also be carried by the retroviral vector.

T Cell Amplification

It is thought that in addition to B cells, other cell types are able to present superantigens, including T cells (Janeway, Current Biology, 1, (1991); Goodglick and Braun, (1994)). It is also known that T cells may present superantigens to other T cells thereby causing the stimulation of the presenting T cells. According to one embodiment of the invention retroviral vectors carrying Sag may also be used to amplify T cells carrying T cells relevant therapeutic genes, in an analogous fashion to that described for B cells.

The present invention provides novel recombinant DNA vectors for gene therapy including a transcriptional unit for the negative acting factor of MMTV to downregulate the expression of heterologous promoters, in particular HIV and MLV promoters.

In a further embodiment the invention provides novel recombinant DNA vectors for gene therapy including both a transcriptional unit for the superantigen activity of MMTV and a B or T cell specific therapeutic peptide or regulatory sequence for the treatment of diseases associated with B or T cells.

Cloning of a Plasmid Allowing the Expression of Sag in Eukaryotic Cells

Plasmid pSVorfexp (Gunzburg et al., Nature, 364:154-158 (1993)) contains the complete 3′ LTR of the MMTV strain Mtv-2 including the full-length sag-ORF (Fasel, et al., EMBO J., 1:2-7 (1982); Genbank Accession NO. V01175) in a HindIII-EcoRI fragment of 1911 kb. This fragment has been joined with a 3404 bp fragment using the HindIII and EcoRI restrictions sites within the multiple cloning site of the plasmid pZeoSV (Invitrogen). In the resulting plasmid pZeoSVorf the SV40 promoter controls the expression of the sag open reading frame. The plasmid further contains a zeocin resistance gene under the control of the major immediate early promoter of the human cytomegalovirus.

Expression of Sag from pZeoSVorf in Mammalian Cells

Plasmid pZeoSVorf was introduced in the murine B-cell lymphoma line A20 (Kim, et al., J. Immunol., 122:549-554 (1979); ATCC No. TIB-208) by electroporation at 1.8 kV (BioRad Gene Pulser). In order to select for cells trasnfected with pZeoSVorf the transfected cells were cultivated with zeocin added to a concentration of 300 μg/ml. The stably transfected cell line obtained was termed A20-SVorf.

To check whether the transfected cells expressed MMTV-Sag, a mixed lymphocyte reaction (MLR) was performed. A20-SVorf cells were growth arrested by irradiation (3000 rad). 1×10⁷ of the cells were co-cultivated with 2×10⁶ primary T-cells freshly isolated from popliteal lymph nodes from BALB/c mice. After 4 days of culture surviving T-cells were stained with R-phycoerythrin-labelled anti-CD3 mAb and either FITC-conjugated anti-Vβ8 mAb or anti-Vβ14 mAb and analyzed by FACS (Elite, Coulter Inc.).

If functional Mtv-2 Sag is expressed, the proliferation of Vβ14−CD3+ cells is stimulated whereas the proliferation of the remaining CD3+ sub-classes is not influenced. In the MLR with the cell line A20-SVorf the percentage of VP-CD3+ increased from 11% to 55.9%. The remaining CD3+ cells did not proliferate and thus show an actual decrease. Thus, it could be demonstrated that the cell line A20-SVorf expressed functional Mtv-2 superantigen.

Construction of Retroviral Vectors Expressing Sag

The retroviral vector plasmid pLXlacZ is based upon plasmid pBAG (Price et al., Proc. Natl. Acad. Sci., USA, 84:156-160 (1987)) from which a 7955 bp AatII-BsrGI fragment was excised, blunt ended and joined with a blunt ended 3529 bp AflIII-EcoRI fragment of plasmid pLXSN (Miller and Rosman, BioTechniques, 7:980-990 (1989): Genbank Accession No. M28248). The plasmid pLXlacZ comprises a neomycin resistance gene and a beta-galactosidase gene flanked by the 5′ LTR of the moloney murine sarcoma virus (MoMuSV) and the 3′ LTR of the moloney murine leukemia virus (MoMuLV). The extended packaging signal sequence containing parts of the Pr65 gag has been mutated to prevent recombination events. Even if recombination occurs it should not result in gag protein production. All other protein coding viral sequences have been deleted. In order to construct a retroviral plasmid expressing the Mtv-2 sag gene pLXlacZ has been cleaved with HindIII and NaeI to remove a 1269 bp fragment containing the neomycin resistance gene. The remaining 10223 bp fragment was ligated to a 1446 bp HindIII-Ecl136 fragment of plasmid pSVorfexp (see above) comprising the full length sag-ORF. The resulting plasmid was termed pLXlacZorf. In this construct the sag-ORF is under control of the SV-40 promoter. The sag gene and the beta-galactosidase gene are flanked by the viral LTRs.

To check whether plasmid pLXlacZorf expresses biologically active sag this plasmid was introduced by electroporation at 1.8 kV into A20 cells and subjected to a mixed lymphocyte reaction (see above). It was observed that the percentage of Vβ14⁺-CD3⁺ cells increased from 11% to 41%. In mock transfected cells and in cells transfected with a plasmid not containing the sag gene, no increase of the percentage of Vβ14⁺-CD3⁺ cells was observed. Thus, the cells transfected with pLXlacZorf express functional Sag.

Construction Of pLXlacZorf Amphotropic Retroviruses

In order to check whether infectious retrovirus are generated the adherent amphotropic packaging cell line PA317 (Miller and Buttimore, Mol. Cell Biol., 6:2895-2902 (1986); ATCC No. CRL-9078) was transfected with plasmid pLXlacZorf. The transfected cells were cocultivated with the B-cell lymphoma line A20. Because the pLXlacZorf plasmid contained no selection marker A20 cells expressing the beta-galactosidase marker gene were harvested by FACS-sorting and further screened for Sag expression using the mixed lymphocute reaction (see above). An increase in the percentage of Vβ14⁺-CD3⁺ cells could be observed.

While this invention has been particularly shown and described with references to preferred embodiments thereof, it will be understood by those skilled in the art that various changes in form and details may be made therein without departing from the scope of the invention encompassed by the appended claims.

What is claimed is:

1. A recombinant vector comprising, in operable linkage,
a) a nucleotide sequence of or corresponding to at least a portion of a vector, which portion is capable of infecting and directing the expression of a coding sequence in target cells; and
b) one or more coding sequences wherein at least one sequence encodes a peptide selected from the group consisting of: a peptide with Sag activity and a derivative of the peptide with Sag activity; and
c) optionally at least one sequence encoding a peptide selected from the group consisting of: a therapeutic peptide and a non-therapeutic peptide.

2. A recombinant vector according to claim 1, wherein said vector is a viral vector selected from the group consisting of: RNA virus vectors, DNA virus vectors and plasmid viral vectors.

3. A recombinant vector according to claim 2 wherein the plasmid viral vector is a eucaryotic expression vector.

4. A recombinant vector according to claim 2, wherein said virus vector is selected from the group consisting of: adenovirus vectors, adenovirus associated virus vectors, herpes virus vectors and retrovirus vectors.

5. A recombinant retroviral vector which is capable of undergoing promoter conversion and is replication-defective comprising, in operable linkage,
a) a 5′ long terminal repeat region comprising the structure U3-R-U5;
b) one or more coding sequences wherein at least one sequence is selected from sequences encoding a peptide selected from the group consisting of: a peptide with Sag activity, and a derivative of the peptide with Sag activity;
c) optionally, at least one sequence encoding a peptide selected from the group consisting of: β-galactosidase, neomycin, alcohol dehydrogenase, puromycin, hypoxanthine phosphoribosyl transferase (HPRT), hygromycin, secreted alkaline phosphatase, Herpes Simplex Virus thymidine kinase, cytosine deaminase, guanine phosphoribosyl transferase (gpt), cytochrome P 450, cell cycle regulatory proteins, tumor suppressor proteins, antiproliferation proteins, and cytokines; and
d) a 3′ long terminal repeat region comprising a completely or partially deleted U3 region wherein said deleted U3 region is replaced by a polylinker sequence carrying at least one unique restriction site.

6. The recombinant vector of claim 5 wherein one or more heterologous DNA fragments are inserted into said polylinker sequence, followed by the R and U5 region.

7. The recombinant vector according to claim 6 wherein said heterologous DNA fragment comprises at least one non-coding sequence selected from regulatory elements or promoters which regulate the expression of at least one of the coding sequences of said recombinant vector.

8. Use of a recombinant vector according to claim 1 for specific amplification of B- or T-cells.

9. A recombinant retroviral vector system comprising a retroviral vector according to claim 5 and a packaging cell line harboring at least one retroviral or recombinant retroviral construct coding for proteins required for said retroviral vector to be packaged.

10. A retroviral provirus produced by the replication of a retroviral vector in the retroviral vector system according to claim 9 comprising:
a) the U3 region which duplicated during the process of reverse transcription in the infected target cell and appears in the 5′ long terminal repeat and in the 3′ long terminal repeat of the resulting provirus, and
b) the U5 of the 5′ long terminal repeat which duplicated during the process of reverse transcription in the infected target cell and appears in the 3′ long terminal repeat and in the 5′ long terminal repeat of the resulting provirus.

11. The retroviral provirus of claim 10 wherein one or more heterologous DNA fragments are inserted into said polylinker sequence, followed by the R and U5 region.

12. mRNA transcribed of a retroviral provirus according to claim 10.

13. A retroviral particle produced by transfecting a packaging cell line according to claim 9 with a retroviral vector, and isolating said retroviral particle.

14. A method for introducing nucleotide sequences encoding peptides with Sag activity into a cell comprising:
a) transfecting a packaging cell line of a retroviral vector system according to claim 9 with a retroviral vector, and
b) infecting the cell with said recombinant retroviruses produced by the packaging cell line.

15. The method of claim 14 wherein the cell is selected from the group consisting of: an animal cell and a human cell.

16. A method for introducing nucleotide sequences encoding peptides with Sag activity into a mammal comprising:
a) transfecting a packaging cell line of a retroviral vector system according to claim 9 with a retroviral vector, and
b) infecting the mammal with said recombinant retroviruses produced by the packaging cell line.

17. A host cell infected with a retroviral vector or a derivative thereof according to claim 10.
